(19) United States
(12) Patent Application Publication
(10) Pub. No.: US 2025/0065012 A1
(43) Pub. Date: Feb. 27, 2025

(54) DIRECTIONAL AND TEMPORAL RELEASE OF DRUGS FROM MEDICAL DEVICES

(71) Applicant: NANO THERAPEUTICS PRIVATE LIMITED, Surat, Gujarat (IN)

(72) Inventors: Rajesh VAISHNAV, Surat (IN); Achyut KHIRE, Surat (IN); Rohit BHANDERI, Surat (IN); Aleesha VAHAB, Surat (IN)

(73) Assignee: NANO THERAPEUTICS PRIVATE LIMITED, Surat, Gujarat (IN)

(21) Appl. No.: 18/726,090

(22) PCT Filed: Dec. 28, 2022
(86) PCT No.: PCT/IN2022/051132

§ 371 Date: Jul. 1, 2024

(30) Foreign Application Priority Data
Jan. 2, 2022 (IN) 202121029892

(51) Int. Cl. A61L 27/34; A61F 2/91; A61L 27/16; A61L 27/18; A61L 27/54; A61L 31/04; A61L 31/06; A61L 31/10; A61L 31/16

(52) U.S. Cl. CPC A61L 27/34; A61F 2/91; A61L 27/16; A61L 27/18; A61L 27/54; A61L 31/048; A61L 31/06; A61L 31/10; A61L 31/16; A61F 2250/0068

(57) ABSTRACT

The present disclosure relates to multilayer polymeric systems for directional and temporal release of drugs from medical devices. Incorporation of drugs in the chosen layer within the multilayer polymeric systems enables release of the drug in the desired direction and over the desired time frame. When more than one drug is delivered, the direction, duration and release rate of each drug can be controlled independent of the other. These multilayer polymeric systems can be used as such or mounted on a variety of drug eluting coronary as well as peripheral stent systems such as carotid artery, hepatic artery, iliac artery, femoral artery, popliteal artery and renal artery stenting. The methods of fabrication are also described.

### CROSS REFERENCE TO RELATED APPLICATIONS

The present disclosure derives its priority from the provisional application titled “Directional and temporal release of drugs from medical devices” filed on 2 Jan. 2022 which is incorporated herein by reference in its entirety.

### FIELD OF THE INVENTION

The present disclosure relates to directional and temporal release of drugs from biomedical devices bearing multiple polymer layers. Specifically, it relates to tailoring the release of drugs over desired time periods to the preferred regions in the vicinity of a stent. More specifically it relates to minimizing/inhibiting the release of the drugs to the regions in the vicinity of a stent, where its presence is not needed and/or may be undesirable over specific time periods, for optimum performance of the stent.

### BACKGROUND OF THE INVENTION

Balloon angioplasty procedures also referred to as percutaneous transluminal coronary angioplasty (PTCA), were introduced in the early 1960s to replace the more invasive bypass surgery wherever possible, as a treatment for occluded arteries. However, the patients undergoing the procedure often experienced vessel reclosure. The vessel closure could be short term (acute reocclusion) and/or long term (restenosis). Restenosis could be the result of mechanical reflex i.e., elastic rebound of the arterial wall and/or the injury to the vessel wall and/or natural healing reaction to the injury of the arterial wall caused by the angioplasty. Net result of the two processes is intimal hyperplasia which may result in the vessel's occlusion. Restenosis could take place within three to six months due to either thrombosis or abnormal tissue growth. In all restenosis following the balloon angioplasty was one of the major issues that needed to be addressed.

Bare metal stents (BMS) introduced in 1980s, partly addressed the restenosis problem. BMS were intended to provide long-term support for the expanded vessel, to keep it from restenosing over time. This often resulted in thrombus formation. The thrombi formed on stents could disengage from the stent, which could result in occlusions elsewhere in the vasculature. To overcome this the patients were subjected to aggressive anti-thrombogenic, and anti-platelet regimes involving the administration of aspirin and clopidogrel for up to 2 months However anti-thrombogenic regime compromises a patient's ability to heal injuries that accompany the stenting procedure or other collateral procedures that may have been required. This called for methods to eliminate the aggressive antithrombogenic therapy associated with coronary stent placement.

Drug Eluting Stents (DES) were introduced to mitigate the situation by controlling the proliferation of active endothelial cells (ECs), accelerating their migration and covering the stent surface. When the ECs formed a continuous, closely packed film, they released nitrous oxide (NO) and hindered the proliferation of SMCs. Another objective of the drug eluting stents was to inhibit the proliferation of smooth muscle cells (SMCs) which was achieved by the local release of an anti-proliferative agent such as Paclitaxel or an immunosuppressive agent such as Sirolimus from the surface of the stent.

However, use of drug eluting stents often led to re-clotting of the artery due to thrombosis after interruption of Dual antiplatelet therapy (DAPT) and DAPT had to be extended for three to twelve months or more. The patient being treated with DAPT needed continuous monitoring. This also created problems when the DAPT had to be discontinued for other medical conditions.

Amongst other reasons, late-stage thrombosis is attributed to incomplete coverage of the stent by ECs, leaving metallic surfaces or polymeric coatings in contact with the blood for extended time periods during which platelet adhesion could occur and lead to formation of a thrombus. It could also be due to the incomplete release of the drug from the drug layer, which would inhibit the proliferation of ECs in their attempts to migrate and cover the surface of the stent and coated layer. The stent strut thickness could also hinder the proliferation of ECs. The rate of proliferation of ECs is negatively correlated with the height of obstacles that they have to overcome. Increasing the thickness of the coating layer results in an overall increase in the thickness of the coated stent and may lead to problems of cracking, flaking, or dislodging of the coating from the stent surface. A summary of the prior efforts to address these issues is presented below.

U.S. Pat. No. 5,873,313 describes spray coating of medical devices with micro-particles of heparin using a pressurized airbrush.

U.S. Pat. No. 5,716,981 discloses a stent coated with a polymer carrier and Paclitaxel. U.S. Pat. Nos. 6,479,654; 6,475,779; and 6,363,938 describe the use of stents to deliver angiogenic agents. U.S. Pat. Nos. 6,071,514 and 5,383,928 disclose the delivery of antithrombotic (antiplatelet) agents. U.S. Pat. Nos. 6,071,514 and 5,383,928. U.S. Pat. No. 6,273,908 discloses the delivery of anticoagulants.

U.S. Pat. No. 6,663,662 describes a multilayer medical device coating incorporating a polymeric diffusion barrier layer for reducing the elution rate of the drug incorporated therein.

U.S. Patent Publication US 2002/0082680 describes an expandable medical device having multiple layers comprising a drug stacked within an opening in a strut. Each layer contains drug particles of different sizes to adjust the release rate of the drug from the device.

U.S. Pat. No. 6,770,729 discloses a medical device coating comprising a polymer and a bioactive material to enable its controlled release from the coating layer.

US Patent Publication 2005/0095267 describes implantable medical devices having nanoparticle drug coatings to improve the solubility of the drug.

U.S. Patent Publication 20050010170 describes applying the first homogeneous solution comprising a drug and a polymer to the implantable medical device, followed by a second homogeneous solution comprising a drug and a polymer on to it. The drug concentration in the two polymer solutions is different.

U.S. Patent Publication 2004/0073294 describes systems and methods for loading a drug into holes of a stent from the drug solution. The filled stent is dried in an oven, and then a next deposit is applied in a similar manner to achieve the desired drug release profile.

U.S. Patent Publication 2006/0222755 discloses loading a drug into holes in a stent wherein the drug is in the form of a thin film which is loaded by punching. A multilayer sheet of drugs could be formed by incorporating layers of drug, drug/polymer, and polymer. The multilayer sheet can be formed with layers of different compositions or different concentrations of the same drug in the layers or by incorporating different drugs in each layer as to release different drugs at different times.

U.S. Pat. No. 7,169,179 discloses a stent with openings for directional delivery of multiple drugs to blood vessels. The delivery of different drugs such as antirestenotic, antithrombotic, antiplatelet, antiproliferative, antineoplastic, immunosuppressive, angiogenic, anti-inflammatory, or antiangiogenic in different quantities, in different directions and at different rates, for delivering agents and/or vasodilators to a blood vessel was disclosed.

U.S. Patent Publication 2011/0045055 discloses implantable or insertable medical devices which can delay the release of one or more drugs for a predetermined time after the device is implanted. This is achieved by incorporating a temporary barrier layer which initially permits little to no release of the drug, followed by the release of the drug according to a predetermined rate as a result of the rupture of the barrier layer.

U.S. Pat. No. 7,927,650 described the problems associated with the use of coatings for the delivery of drugs from stents. The surface coatings can provide little actual control over the drug release kinetics. The coatings are very thin typically 5-8μ. The surface area of the stent is by comparison very large so that the drug has very short diffusion path to release into the surrounding tissue. Increasing the thickness of the coating could provide a better control over the release kinetics, enable higher drug loading but bears the risk of cracking, flaking, and dislodging the coating from the stent surface. The patent disclosed loading of drugs in the powder form into the holes of a stent device followed by treating the same with a solvent to ensure adhesion of the drug in the holes.

U.S. Pat. No. 8,734,829 discloses a medical device containing a substrate, a region over the substrate containing a drug a nanoporous polymeric layer disposed over the drug containing region and a microporous non-polymeric layer disposed over the nanoporous polymeric layer.

U. S. Patent Publication 2014/0288122 discloses a method for inhibiting platelet aggregation in a patient by administering to the patient a bolus injection of Tirofiban about 25 μg/kg, and administering to the patient, after the bolus injection, an intravenous infusion for a period of between about 12 hours and about 72 hours of Tirofiban at the rate of about 0.15 μg/kg/min.

U.S. Pat. No. 8,932,345 discloses medical device coatings that release a drug at different rates from different regions of the medical device coating. Particles comprising a drug in two or more different particles sizes are incorporated within a single layer on the surface of the implantable device. The drug concentration is higher in the first region of the coating than in the second region of the coating. Such coatings are formed by processes wherein the droplet size of a spray coating solution is changed during the coating process.

U. S. Patent Publication 2021/0361449 describes drug eluting stents, methods of making, using, and varying the long-term stability of the drug eluting stents, which may include a stent framework; a drug-containing layer; a drug embedded in the drug-containing layer; and a biocompatible base layer disposed over the stent framework and supporting the drug-containing layer. The thickness of the drug-containing layer may vary. The drug containing layer may dissolve between 45 and 60 days after stent implantation.

From a clinical standpoint, clot formation in the luminal region could result from acute stent thrombosis which occurs within hours of stent implantation and sub-acute as well as late stent thrombosis which may occur up to thirty days or longer after stent implantation. While dual antiplatelet therapy could be administered to overcome thrombosis, it is known to lead to increased bleeding. DAPT is reportedly less effective in addressing the issue in the case of intracranial biomedical devices. Some patient populations are resistive to DAPT.

Tirofiban is a platelet GP IIb/IIIa inhibitor and a powerful anti-platelet aggregation drug. After systemic administration, platelet aggregation can be inhibited up to 96%, which can reduce the incidence of Major adverse cardiac events (MACE/MACCEs), however it increases risk of bleeding. Intracoronary injection of Tirofiban prevents platelet aggregation as well as microcirculation dysfunction during stenting as well as delayed percutaneous coronary intervention (PCI) in Acute myocardial intervention (AMI) patients. A sustained release of Tirofiban HCl locally through a biomedical device in the luminal region would prevent acute, sub-acute and late thrombosis as well as bleeding risk.

Dysfunctional vascular endothelium leads to in stent restenosis in the absence of antithrombotic and antiatherogenic properties (agents). This endothelial dysfunction triggers vascular smooth muscle cells (VMSCs) proliferation. As a result, VMSCs overgrow, which leads to the blockage of vessels over time. While the drug eluting stents lower the rate of restenosis vis a vis bare metal stents, there is a concern that DES may be associated with a higher risk of late and very late stent thrombosis, especially in the absence of DAPT or when DAPT is discontinued.

Sirolimus is an immunosuppressant that suppresses VMSCs proliferation. A sustained release of Sirolimus over extended time periods in the abluminal region would help restrict VMSCs overgrowth into the blood vessels. This could be achieved by limiting the loss of Sirolimus into the luminal region. Thus, there is a need for directional and temporal release of both Tirofiban and Sirolimus from biomedical devices. Such a delivery system would find further applications in other coated biomedical devices for the release of anti-thrombotic, vasodilators, Vasorelaxants in luminal region and anti-proliferative agents, anti-inflammatory, steroids, vasodilators, vasorelaxants, lipid lowering agents in the abluminal region.

### SUMMARY OF THE INVENTION

It has now been surprisingly found that the directional and temporal release of drugs can be achieved by multiple polymer layer coated stents. More particular, it has been found that the release of a drug can be minimized or inhibited in the regions where its presence is not needed or may be undesirable. This helps in extending the duration of release of the drug into the regions where its presence is desirable. Further, the release kinetics of drugs from multiple polymer layers can be controlled. Various embodiments of the disclosure are now described below, which illustrates the disclosure but does not limit the scope of the disclosure:
1. According to an embodiment of the disclosure, the multiple polymer layer coated biomedical device comprises at least one hydrophilic drug and one hydrophobic drug, wherein the hydrophobic drug is contained in at least one hydrophobic polymer layer.
2. According to an embodiment of the disclosure, the multiple polymer layer coated biomedical device is selected from stents, grafts, balloons, catheters, filters or mesh like structures, other similar endovascular devices.
3. According to an embodiment of the disclosure, the multiple polymer layer coated stent of the disclosure comprises a bilayer formed by coating two hydrophobic polymers successively, wherein the release of a water-soluble drug is controlled by the molecular weight (as reflected in the intrinsic viscosity of the polymer) in which the said drug is incorporated.
4. According to an embodiment of the disclosure, the multiple polymer layer coated stent of the disclosure comprises a bilayer formed by coating a hydrophilic polymer containing a water-soluble drug and a hydrophobic polymer, wherein the release of the drug is controlled by the molecular weight (as reflected in the K value of the hydrophilic polymer).
5. According to an embodiment of the disclosure, the multiple polymer layer coated stent of the disclosure comprises a bilayer formed by coating two hydrophobic polymers, wherein the release of a water-soluble drug is modified by the hydrophobicity of the polymer in which the drug is incorporated.
6. According to an embodiment of the disclosure, the multiple polymer layer coated stent of the disclosure comprises a bilayer formed by coating a hydrophobic polymer followed by a hydrophobic polymer containing a hydrophobic drug wherein the hydrophobic drug is preferentially released in the abluminal region.
7. According to an embodiment of the disclosure, the multiple polymer layer coated stent of the disclosure comprises a bilayer formed by coating a hydrophobic polymer followed by a hydrophobic polymer containing a hydrophobic drug wherein the hydrophobic drug cumulatively released in the luminal region is less than 5% in 6 hrs.
8. According to an embodiment of the disclosure the multiple polymer layer coated stent of the disclosure comprises a bilayer formed by coating a hydrophobic polymer followed by a hydrophobic polymer containing a hydrophobic drug wherein the hydrophobic drug cumulatively released in the luminal region is less than 6% in 24 hrs.
9. According to an embodiment of the disclosure the multiple polymer layer coated stent of the disclosure comprises a bilayer formed by coating a hydrophobic polymer followed by a hydrophobic polymer containing a hydrophobic drug wherein the hydrophobic drug cumulatively released in the luminal region is less than 20% in 14 days.
10. According to an embodiment of the disclosure the multiple polymer layer coated stent of the disclosure comprises a bilayer formed by coating a hydrophobic polymer followed by hydrophobic polymer containing a hydrophobic drug wherein the cumulative release of the hydrophobic drug in the over a period of 24 days is around 50%.
11. According to an embodiment of the disclosure the multiple polymer layer coated stent of the disclosure comprises a bilayer formed by coating a hydrophobic polymer followed by a blend of two hydrophobic polymer containing a hydrophobic drug wherein the cumulative release of the hydrophobic drug over a period of 24 days is 75%.
12. According to an embodiment of the disclosure the multiple polymer layer coated stent of the disclosure comprises a bilayer formed by coating a hydrophobic polymer followed by blend of hydrophobic polymer and hydrophilic polymer containing a hydrophobic drug wherein the cumulative release of the hydrophobic drug over a period of 24 days is 77%.
13. According to an embodiment of the disclosure the multiple polymer layer coated stent of the disclosure comprises a bilayer formed by coating a hydrophilic drug in a hydrophobic polymer layer followed by a hydrophobic polymer layer wherein the hydrophilic drug cumulatively released in the abluminal region in 24 hrs is less than 10%.
14. According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein less than 1% of hydrophobic drug is cumulatively released in the luminal region up to 24 hrs.
15. According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein hydrophilic drug is preferentially released in a luminal region.
16. According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein 32-74% of hydrophobic drug is released cumulatively in the abluminal region in 21 days.
17. According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein 21-28% of hydrophobic drug is released cumulatively in the luminal region in 21 days.
18. According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein 68-98% of hydrophilic drug is released cumulatively in the luminal region in 9 days.
19. According to an embodiment of the disclosure the multiple polymer layer coated stent of the disclosure comprises a tri layer formed by coating a composition comprising a hydrophilic drug and a hydrophobic polymer, followed by a hydrophobic polymer followed by a hydrophobic polymer containing a hydrophobic drug wherein 40-46% hydrophilic drug is released cumulatively in the luminal region in 30 days.
20. According to an embodiment of the disclosure the multiple polymer layer coated stent of the disclosure comprises a tri layer formed by coating a composition comprising a hydrophilic drug and a hydrophobic polymer, followed by a hydrophobic polymer followed by a hydrophobic polymer containing a hydrophobic drug wherein 40-65% hydrophobic drug is released cumulatively in the abluminal region in 30 days.
21. According to an embodiment of the disclosure the multiple polymer layer coated stent of the disclosure comprises a tri layer formed by coating a composition comprising a hydrophilic drug and a hydrophilic polymer, followed by a composition comprising a hydrophilic drug and a hydrophobic polymer, followed by a hydrophobic polymer containing a hydrophobic drug wherein no hydrophilic drug is released in the abluminal region.
22. According to an embodiment of the disclosure the multiple polymer layer coated stent of the disclosure comprises four layers formed by coating a composition comprising a hydrophilic drug and a hydrophilic polymer, followed by a composition comprising a hydrophilic drug and a hydrophobic polymer, followed by a hydrophobic polymer layer, followed by a layer containing a hydrophobic drug and a hydrophobic polymer wherein the hydrophobic drug is not released in the luminal region up to at least 24 hrs.
23. According to an embodiment of the disclosure the multiple polymer layer coated stent of the disclosure comprises four layers formed by coating a composition comprising a hydrophilic drug and a hydrophilic polymer, followed by, composition comprising a hydrophilic drug and a hydrophobic polymer, followed by a hydrophobic polymer layer, followed by a layer containing a hydrophobic drug and a hydrophobic polymer wherein 75% hydrophobic drug is released cumulatively over 30 days in abluminal region.
24. According to an embodiment of the disclosure the multiple polymer layer coated stent of the disclosure comprises four layers formed by coating a composition comprising a hydrophilic drug and a hydrophilic polymer, followed by, composition comprising a hydrophilic drug and a hydrophobic polymer, followed by a hydrophobic polymer layer, followed by a layer containing a hydrophobic drug and a hydrophobic polymer wherein 64% of hydrophilic drug is released cumulatively in the luminal region in 30 days.
25. According to an embodiment of the disclosure the multiple polymer layer coated stent of the disclosure comprises four layers formed by coating a composition comprising a hydrophilic drug and a hydrophilic polymer, followed by, composition comprising a hydrophilic drug and a hydrophobic polymer, followed by a hydrophobic polymer layer, followed by a layer containing a hydrophobic drug and a hydrophobic polymer wherein the hydrophilic drug in the hydrophilic polymer layer and the hydrophilic drug in the hydrophobic polymer layer are different.
26. According to an embodiment of the disclosure the multiple polymer layer coated stent of the disclosure comprises a bilayer formed by coating a hydrophobic polymer containing a water-soluble drug followed by coating comprising a hydrophobic drug and a blend of hydrophobic polymers wherein the stent administered to a mammal inhibits thrombus formation up to at least ten days.
27. According to an embodiment of the disclosure the multiple polymer layer coated stent of the disclosure comprises a bilayer formed by coating a hydrophobic polymer containing a hydrophilic drug followed by coating comprising a hydrophobic drug and a blend of hydrophobic polymers wherein the stent administered to a mammal inhibits neointimal growth up to at least ten days.
28. According to an embodiment of the disclosure, the coated stent of the disclosure comprises a bilayer formed by coating a hydrophobic polymer containing a water-soluble drug followed by another coating comprising a hydrophobic drug and a blend of hydrophobic polymers wherein the stent administered to a mammal exhibits endothelialization before 10 and 28 days.
29. According to an embodiment of the disclosure, the hydrophilic polymer is polyvinyl pyrrolidone.
30. According to an embodiment of the disclosure, the hydrophilic drug is chosen from the class of antithrombotics, anticoagulants, antiplatelets, vasodilators, vasorelaxants, anti-hypertensives, cells, antibodies, peptides, elastin, and hemocompatibility enhancers.
31. According to an embodiment of the disclosure antithrombotic, selected from Argatroban, Inogatran, Melagatran and their pharmaceutically acceptable derivatives.
32. According to an embodiment of the disclosure, anticoagulant selected from Warfarin/coumarin and its derivatives, Vitamin K antagonists, Heparin and their derivatives, low molecular weight heparin (LMWH) and their derivatives e.g. Bemiparin, Nadroparin, Reviparin, Enoxaparin, Parnaparin, Certoparin, Dalteparin, Tinzaparin, Synthetic pentasugar derivatives (Factor Xa inhibitors) e.g., Fondaparinux, Idraparinux, Idrabiotaparinux.
33. According to an embodiment of the disclosure Antiplatelet drugs, selected from the class of irreversible cyclooxygenase inhibitors e.g., Aspirin, Triflunisal (Disgren); 34. According to an embodiment of the disclosure Antiplatelet drugs, selected from the class of Adenosine diphosphate (ADP) receptor inhibitors e.g., Cangrelor, Clopidogrel, Prasugrel, Ticagrelor, Ticlopidine; 35. According to an embodiment of the disclosure Antiplatelet drugs, selected from the class of Phosphodiesterase inhibitors e.g., Cilostazol.
36. According to an embodiment of the disclosure Antiplatelet drugs, selected from the class of Protease-activated receptor-1 (PAR-1) antagonists e.g., Vorapaxar.
37. According to an embodiment of the disclosure Antiplatelet drugs, selected from the class of Glycoprotein IIB/IIIA inhibitors e.g., Abciximab, Eptifibatide, Tirofiban etc.
38. According to an embodiment of the disclosure Antiplatelet drugs, selected from the class of Adenosine reuptake inhibitors e.g., Dipyridamole.
39. According to an embodiment of the disclosure Antiplatelet drugs, selected from the class of Thromboxane inhibitors/Thromboxane synthase inhibitors e.g., Terutroban
40. According to an embodiment of the disclosure, anti-hypertensive, selected from Diuretics, Beta-blockers. ACE inhibitors, Angiotensin II receptor blockers, Calcium channel blockers. Alpha blockers, Alpha-2 Receptor Agonists.
41. According to an embodiment of the disclosure, Vasodilator, selected from Nitric oxide and its derivatives or precursors, prostaglandins, adenosine, minoxidil, etc., 42. According to an embodiment of the disclosure, Vasorelaxant, selected from Hydralazine, minoxidil.
43. According to an embodiment of the disclosure, hemocompatibility enhancer selected from group of compounds acting on coagulation mechanism, haemolysis mechanism, like heparin surface coating etc.
44. According to an embodiment of the disclosure, the hydrophobic polymer is chosen from poly-ε caprolactone (PCL), poly lactide co-ε caprolactone (PLCL), poly lactic acid (PLA), Poly lactide co-glycolide (PLGA)
45. According to an embodiment of the disclosure, the hydrophobic drug is chosen from the class of antiproliferative, anti-inflammatory, antibiotic, bioactive molecules, vasodilators and vasorelaxants
46. According to an embodiment of the disclosure, antiproliferative selected from antiproliferative/cytostatic/cytostatic chemotherapeutic agents e.g., Rapamycin, everolimus, zotarolimus, paclitaxel etc.,
47. According to an embodiment of the disclosure, Anti-inflammatory, drug selected from Aspirin, naproxen, Cox2 inhibitors e.g., Celecoxib, rofecoxib and steroids e.g., Dexamethasone.,
48. According to an embodiment of the disclosure, Vasodilator, selected from Nitric oxide and its derivatives or precursors, prostaglandins, adenosine, minoxidil, etc.,
49. According to an embodiment of the disclosure, Vasorelaxant, selected from Hydralazine, minoxidil.

### BRIEF DESCRIPTION OF DRAWINGS

The objectives and advantages of the present invention will become apparent from the following description read in accordance with the accompanying drawings wherein,

FIG. 1: Tirofiban·HCl release using rotating bottle apparatus from bilayer coated stents 1a, 1b and 1c of example 1.

FIG. 2: Release of Aspirin, Tirofiban Hydrochloride and Clopidogrel sulfate from rotating bottle apparatus and tube apparatus of bilayer coated stents 2a, 2b and 2c of example 2.

FIG. 3a: Release of Tirofiban·HCl from rotating bottle apparatus and tube apparatus of bilayer coated stents 3a and 3b of example 3.

FIG. 3b: Release of Tirofiban·HCl from a rotating bottle apparatus and tube apparatus of bilayer coated stents 3c and 3d of example 3.

FIG. 4: Release of Sirolimus, Everolimus and Dexamethasone acetate from a rotating bottle apparatus and tube apparatus of bilayer coated stents 4a, 4b, 4c and 4d of example 4.

FIG. 5: Release of Sirolimus, Everolimus and Dexamethasone acetate from a rotating bottle apparatus and tube apparatus of bilayer coated stents 5a, 5b and 5c of example 5 FIG. 6: Release of Everolimus and Sirolimus from a rotating bottle apparatus of bilayer coated stents 6a, 6b and 6c of example 6.

FIG. 7: Release of Dexamethasone acetate from a rotating bottle apparatus and tube apparatus. of bilayer coated stent 7 of example 7.

FIG. 8: Release of Clopidogrel sulphate, Argatroban and Tirofiban·HCl from a rotating bottle apparatus and tube apparatus of bilayer coated stents 8a, 8b and 8c of example 8.

FIG. 9: Sirolimus release from a rotating bottle apparatus and tube apparatus of Tri layer coated stents 9a, 9b, 9c and 9d of example 9

FIG. 10: Release of Tirofiban·HCl and Aspirin from rotating bottle apparatus and tube apparatus of Tri layer coated stents 9a, 9b, 9c and 9d of example 9.

FIG. 11: Release of Tirofiban·HCl from rotating bottle apparatus and tube apparatus of Tri layer coated stents 10a, 10b, 10c and 10d of example 10.

FIG. 12: Release of Sirolimus from rotating bottle apparatus and tube apparatus of Tri layer coated stents 10a, 10b, 10c and 10d of example 10.

FIG. 13: Release of Tirofiban·HCl and Aspirin from rotating bottle apparatus and tube apparatus of Tri layer coated stents 11a, 11b and 11e of example 11.

FIG. 14: Release of Tirofiban Hydrochloride and Sirolimus from a rotating bottle apparatus and tube apparatus of a 4-layer coated stent 12 of example 12

FIG. 15: Release of Tirofiban·HCl, Aspirin and Sirolimus from a rotating bottle apparatus and tube apparatus of a 4-layer coated stent 13 of example 13.

FIG. 16: Release of Sirolimus and Tirofiban from a rotating bottle apparatus and tube apparatus of a single layer coated stent 14 of example 14.

FIG. 17: Release of Sirolimus and Tirofiban·HCl from a rotating bottle apparatus.

FIG. 18: Images of histopathological evaluation of multiple polymer layer coated stent containing Sirolimus and Tirofiban·HCl administered to a pig.

### DESCRIPTION OF THE INVENTION

References in the specification to “one embodiment” or “an embodiment” means that a particular feature, structure, characteristic, or function described in connection with the embodiment is included in at least one embodiment of the invention. The appearances of the phrase “in one embodiment” in various places in the specification are not necessarily all referring to the same embodiment.

References in the specification to “preferred embodiment” means that a particular feature, structure, characteristic, or function described in detail thereby omitting known constructions and functions for clear description of the present invention.

The foregoing description of specific embodiments of the present invention has been presented for purposes of illustration and description. They are not intended to be exhaustive or to limit the present invention to the precise forms disclosed and obviously many modifications and variations are possible in light of the above teaching.

Balloon angioplasty emerged as a less invasive substitute to bypass surgery in many situations. The problems associated with balloon angioplasty were mitigated by bare metal stents and subsequently by drug eluting stents. However, the drug eluting stents still suffer from limitations. Approaches to overcome these have been summarized in the prior art. On this background it has now been surprisingly found that the drug release kinetics can be controlled from multiple polymer layer coated stents. According to the present disclosure, the direction of release is controlled by the choice of hydrophobic polymer layer and its location within the multiple layers. The duration of the drug release is controlled by the choice of the polymer layer in which the drug is incorporated. A detailed description which illustrates the various facets of the disclosure but does not limit it is provided in subsequent paragraphs.

### Definitions

As used herein, ‘directional release’ refers to the release of drug from the surface of a medical device towards a desired direction or one side of the artery, either in the luminal region or abluminal region. In case of multiple drugs, the direction of release may be different or may be the same.

As used herein, ‘temporal release, refers to the controlled release of drug from the surface of a medical device over a desired time frame. When more than one drug is used, the desired time frame will be different for each other. The desired time frame may be different for the same drug in different situations.

As used herein, the phrase “preferentially released” implies that the release of the drug in the desired region is greater than that in another region.

As used herein, ‘hydrophobic polymer’ refers to a polymer which can be coated from a solvent that is immiscible with water.

In case of copolymers of lactic and glycolic acid that contain both hydrophobic and hydrophilic monomers, the copolymer with a higher lactic acid content, is considered more hydrophobic than the copolymer containing lower lactic acid content.

As used herein, ‘hydrophilic polymer refers to a polymer which can be coated from a solvent that is water miscible.

To the person having ordinary skill in the art, it is known that the molecular weight of polymers is directly related to their intrinsic viscosity. In the case of polyvinylpyrrolidone, a higher K value indicates a higher molecular weight.

As used herein, the term hydrophobic drug refers to a drug that is soluble in a solvent that is not miscible with water.

Various embodiments of the disclosure are now described below, which illustrates the disclosure but does not limit the scope of the disclosure:

According to an embodiment of the disclosure, the multiple polymer layer coated biomedical device comprises at least one hydrophilic drug and one hydrophobic drug, wherein the hydrophobic drug is contained in at least one hydrophobic polymer layer.

According to an embodiment of the disclosure, the multiple polymer layer coated biomedical device is selected from stents, grafts, balloons, catheters, filters or mesh like structures, other similar endovascular devices.

According to an embodiment of the disclosure, the multiple polymer layer coated stent of the disclosure comprises a bilayer formed by coating two hydrophobic polymers successively, wherein the release of a water-soluble drug is controlled by the molecular weight (as reflected in the intrinsic viscosity of the polymer) in which the said drug is incorporated.

According to an embodiment of the disclosure, the multiple polymer layer coated stent of the disclosure comprises a bilayer formed by coating a hydrophilic polymer containing a water-soluble drug and a hydrophobic polymer, wherein the release of the drug is controlled by the molecular weight (as reflected in the K value of the hydrophilic polymer).

According to an embodiment of the disclosure, the multiple polymer layer coated stent of the disclosure comprises a bilayer formed by coating two hydrophobic polymers, wherein the release of a water-soluble drug is modified by the hydrophobicity of the polymer in which the drug is incorporated.

According to an embodiment of the disclosure, the multiple polymer layer coated stent of the disclosure comprises a bilayer formed by coating a hydrophobic polymer followed by a hydrophobic polymer containing a hydrophobic drug wherein the hydrophobic drug is preferentially released in the abluminal region.

According to an embodiment of the disclosure, the multiple polymer layer coated stent of the disclosure comprises a bilayer formed by coating a hydrophobic polymer followed by a hydrophobic polymer containing a hydrophobic drug wherein the hydrophobic drug cumulatively released in the luminal region is less than 5% in 6 hrs.

According to an embodiment of the disclosure the multiple polymer layer coated stent of the disclosure comprises a bilayer formed by coating a hydrophobic polymer followed by a hydrophobic polymer containing a hydrophobic drug wherein the hydrophobic drug cumulatively released in the luminal region is less than 6% in 24 hrs.

According to an embodiment of the disclosure the multiple polymer layer coated stent of the disclosure comprises a bilayer formed by coating a hydrophobic polymer followed by a hydrophobic polymer containing a hydrophobic drug wherein the hydrophobic drug cumulatively released in the luminal region is less than 20% in 14 days.

According to an embodiment of the disclosure the multiple polymer layer coated stent of the disclosure comprises a bilayer formed by coating a hydrophobic polymer followed by hydrophobic polymer containing a hydrophobic drug wherein the cumulative release of the hydrophobic drug in the over a period of 24 days is around 50%.

According to an embodiment of the disclosure the multiple polymer layer coated stent of the disclosure comprises a bilayer formed by coating a hydrophobic polymer followed by a blend of two hydrophobic polymer containing a hydrophobic drug wherein the cumulative release of the hydrophobic drug over a period of 24 days is 75%.

According to an embodiment of the disclosure the multiple polymer layer coated stent of the disclosure comprises a bilayer formed by coating a hydrophobic polymer followed by blend of hydrophobic polymer and hydrophilic polymer containing a hydrophobic drug wherein the cumulative release of the hydrophobic drug over a period of 24 days is 77%.

According to an embodiment of the disclosure the multiple polymer layer coated stent of the disclosure comprises a bilayer formed by coating a hydrophilic drug in a hydrophobic polymer layer followed by a hydrophobic polymer layer wherein the hydrophilic drug cumulatively released in the abluminal region in 24 hrs is less than 10%.

According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein less than 1% of hydrophobic drug is cumulatively released in the luminal region up to 24 hrs.

According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein hydrophilic drug is preferentially released in a luminal region.

According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein 32-74% of hydrophobic drug is released cumulatively in the abluminal region in 21 days.

According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein 21-28% of hydrophobic drug is released cumulatively in the luminal region in 21 days.

According to an embodiment of the disclosure the multiple polymer layer coated stent of the disclosure comprises a tri layer formed by coating a composition comprising a hydrophilic drug and a hydrophilic polymer, followed by a hydrophobic polymer followed by a hydrophobic polymer containing a hydrophobic drug wherein 68-98% of hydrophilic drug is released cumulatively in the luminal region in 9 days.

According to an embodiment of the disclosure the multiple polymer layer coated stent of the disclosure comprises a tri layer formed by coating a composition comprising a hydrophilic drug and a hydrophobic polymer, followed by a hydrophobic polymer followed by a hydrophobic polymer containing a hydrophobic drug wherein 40-46% hydrophilic drug is released cumulatively in the luminal region in 30 days.

According to an embodiment of the disclosure the multiple polymer layer coated stent of the disclosure comprises a tri layer formed by coating a composition comprising a hydrophilic drug and a hydrophobic polymer, followed by a hydrophobic polymer followed by a hydrophobic polymer containing a hydrophobic drug wherein 40-65% hydrophobic drug is released cumulatively in the abluminal region in 30 days.

According to an embodiment of the disclosure the multiple polymer layer coated stent of the disclosure comprises a tri layer formed by coating a composition comprising a hydrophilic drug and a hydrophilic polymer, followed by a composition comprising a hydrophilic drug and a hydrophobic polymer, followed by a hydrophobic polymer containing a hydrophobic drug wherein no hydrophilic drug is released in the abluminal region.

According to an embodiment of the disclosure the multiple polymer layer coated stent of the disclosure comprises four layers formed by coating a composition comprising a hydrophilic drug and a hydrophilic polymer, followed by a composition comprising a hydrophilic drug and a hydrophobic polymer, followed by a hydrophobic polymer layer, followed by a layer containing a hydrophobic drug and a hydrophobic polymer wherein the hydrophobic drug is not released in the luminal region up to at least 24 hrs.

According to an embodiment of the disclosure the multiple polymer layer coated stent of the disclosure comprises four layers formed by coating a composition comprising a hydrophilic drug and a hydrophilic polymer, followed by, composition comprising a hydrophilic drug and a hydrophobic polymer, followed by a hydrophobic polymer layer, followed by a layer containing a hydrophobic drug and a hydrophobic polymer wherein 75% hydrophobic drug is released cumulatively over 30 days in abluminal region.

According to an embodiment of the disclosure the multiple polymer layer coated stent of the disclosure comprises four layers formed by coating a composition comprising a hydrophilic drug and a hydrophilic polymer, followed by, composition comprising a hydrophilic drug and a hydrophobic polymer, followed by a hydrophobic polymer layer, followed by a layer containing a hydrophobic drug and a hydrophobic polymer wherein 64% of hydrophilic drug is released cumulatively in the luminal region in 30 days.

According to an embodiment of the disclosure the multiple polymer layer coated stent of the disclosure comprises four layers formed by coating a composition comprising a hydrophilic drug and a hydrophilic polymer, followed by, composition comprising a hydrophilic drug and a hydrophobic polymer, followed by a hydrophobic polymer layer, followed by a layer containing a hydrophobic drug and a hydrophobic polymer wherein the hydrophilic drug in the hydrophilic polymer layer and the hydrophilic drug in the hydrophobic polymer layer are different.

According to an embodiment of the disclosure the multiple polymer layer coated stent of the disclosure comprises a bilayer formed by coating a hydrophobic polymer containing a water-soluble drug followed by coating comprising a hydrophobic drug and a blend of hydrophobic polymers wherein the stent administered to a mammal inhibits thrombus formation up to at least ten days.

According to an embodiment of the disclosure the multiple polymer layer coated stent of the disclosure comprises a bilayer formed by coating a hydrophobic polymer containing a hydrophilic drug followed by coating comprising a hydrophobic drug and a blend of hydrophobic polymers wherein the stent administered to a mammal inhibits neointimal growth up to at least ten days.

According to an embodiment of the disclosure, the coated stent of the disclosure comprises a bilayer formed by coating a hydrophobic polymer containing a water-soluble drug followed by another coating comprising a hydrophobic drug and a blend of hydrophobic polymers wherein the stent administered to a mammal exhibits endothelialization before 10 and 28 days.

According to an embodiment of the disclosure, the hydrophilic polymer is polyvinyl pyrrolidone.

According to an embodiment of the disclosure, the hydrophilic drug is chosen from the class of antithrombotics, anticoagulants, antiplatelets, vasodilators, vasorelaxants, anti-hypertensives, cells, antibodies, peptides, elastin, and hemocompatibility enhancers.

According to an embodiment of the disclosure antithrombotic, selected from Argatroban, Inogatran, Melagatran and their pharmaceutically acceptable derivatives.

According to an embodiment of the disclosure, anticoagulant selected from Warfarin/coumarin and its derivatives, Vitamin K antagonists, Heparin and their derivatives, low molecular weight heparin (LMWH) and their derivatives e.g. Bemiparin, Nadroparin, Reviparin, Enoxaparin, Parnaparin, Certoparin, Dalteparin, Tinzaparin, Synthetic pentasugar derivatives (Factor Xa inhibitors) e.g., Fondaparinux, Idraparinux, Idrabiotaparinux.

According to an embodiment of the disclosure Antiplatelet drugs, selected from the class of irreversible cyclooxygenase inhibitors e.g., Aspirin, Triflunisal (Disgren);

According to an embodiment of the disclosure Antiplatelet drugs, selected from the class of Adenosine diphosphate (ADP) receptor inhibitors e.g., Cangrelor, Clopidogrel, Prasugrel, Ticagrelor, Ticlopidine;

According to an embodiment of the disclosure Antiplatelet drugs, selected from the class of Phosphodiesterase inhibitors e.g., Cilostazol.

According to an embodiment of the disclosure Antiplatelet drugs, selected from the class of Protease-activated receptor-1 (PAR-1) antagonists e.g., Vorapaxar.

According to an embodiment of the disclosure Antiplatelet drugs, selected from the class of Glycoprotein IIB/IIIA inhibitors e.g., Abciximab, Eptifibatide, Tirofiban etc.

According to an embodiment of the disclosure Antiplatelet drugs, selected from the class of Adenosine reuptake inhibitors e.g., Dipyridamole.

According to an embodiment of the disclosure Antiplatelet drugs, selected from the class of Thromboxane inhibitors/Thromboxane synthase inhibitors e.g., Terutroban

According to an embodiment of the disclosure, anti-hypertensive, selected from Diuretics, Beta-blockers. ACE inhibitors, Angiotensin II receptor blockers, Calcium channel blockers. Alpha blockers, Alpha-2 Receptor Agonists.

According to an embodiment of the disclosure, Vasodilator, selected from Nitric oxide and its derivatives or precursors, prostaglandins, adenosine, minoxidil, etc.,

According to an embodiment of the disclosure, Vasorelaxant, selected from Hydralazine, minoxidil.

According to an embodiment of the disclosure, hemocompatibility enhancer selected from group of compounds acting on coagulation mechanism, haemolysis mechanism, like heparin surface coating etc.

According to an embodiment of the disclosure, the hydrophobic polymer is chosen from poly-ε caprolactone (PCL), poly lactide co-ε caprolactone (PLCL), poly lactic acid (PLA), Poly lactide co-glycolide (PLGA)

According to an embodiment of the disclosure, the hydrophobic drug is chosen from the class of antiproliferative, anti-inflammatory, antibiotic, bioactive molecules, vasodilators and vasorelaxants.

According to an embodiment of the disclosure, antiproliferative selected from antiproliferative/cytostatic/cytostatic chemotherapeutic agents e.g., Rapamycin, everolimus, zotarolimus, paclitaxel etc.

According to an embodiment of the disclosure, Anti-inflammatory, drug selected from Aspirin, naproxen, Cox2 inhibitors e.g., Celecoxib, rofecoxib and steroids e.g., Dexamethasone.

According to an embodiment of the disclosure, Vasodilator, selected from Nitric oxide and its derivatives or precursors, prostaglandins, adenosine, minoxidil, etc.

According to an embodiment of the disclosure, Vasorelaxant, selected from Hydralazine, minoxidil.

### Procedure for Drug Coating

1. A clean L605 electro polished stent of size 2.75×12 mm (OD×Length) was taken out using a hypodermic needle from the vial and recorded the initial weight before mounting it between two collates of the coating machine.
2. The solution was poured into the spray gun cup, the coating machine was started and monitored the coating solution flow.
3. Stent was carefully removed from collates after complete coating. The coated stent should be handled in such a way that coating was not damaged. The coated stent was placed on the weighing balance pan, to measure its weight.
4. The stent was then placed in respective vial and kept in vacuum oven at −27±2 Hg and room temperature for minimum 12 Hrs for drying purpose. A hole was made on the vial cap so that vacuum could be created inside the vial.
5. After the drying process, the stent was taken out from vacuum oven and weighed
6. For the abluminus layer coating, the stent was mounted on a mandrel and repeated the steps 2, 3, 4 and 5.

### Procedure for Drug Release in Tube

A coated stent, crimped over the balloon was inserted into a silicone tube. It was expanded by applying pressure using an inflation device. This tube was immersed in a glass tube containing 4 ml phosphate buffered saline of pH 7.4, used as the release medium. The glass tube was loaded into the dissolution apparatus. The equipment was set at 10 RPM and 250° C. 1 ml of the samples from the release medium were withdrawn at 0.25 h, 0.5 h, 1 h, 3 h, 6 h, 24 h, 48 h and multiple equidistant timepoints thereafter, till no further release was observed. 1 ml buffer medium was replaced immediately after each sampling. The amount of drug in 4 ml was calculated, from HPLC analysis of the aliquot using a UV detector. Subsequently, the cumulative amount of drug release and % drug release at each time point was calculated.

Drug release for Tirofiban·HCl was monitored by recording absorbance at 226 nm, Aspirin at 240 nm, Sirolimus at 276 nm, Everolimus at 277 nm, Clopidogrel sulphate at 240 nm, Dexamethasone acetate at 254 nm and Argatroban at 254 nm.

### Procedure for Drug Release in Rotating Bottle Apparatus

A coated crimped stent was inserted in the glass tube containing 4 ml release medium (Phosphate buffered saline of pH 7.4) and the stent was expanded by applying pressure using an inflation device. Glass tube was loaded in the rotating bottle apparatus. The equipment was set at 10 RPM and 250° C. 1 ml sample from the release medium was withdrawn at 0.25 h, 0.5 h, lh, 3 h, 6 h, 24 h, 48 h and every 24 h thereafter, till no further release was observed. 1 ml buffer medium was replaced immediately after each sampling. The amount of drug in 4 ml was calculated, from HPLC analysis of the aliquot using a UV detector. Subsequently, the cumulative amount of drug release and % drug release at each time point was calculated.

The drug released under this experimental condition (rotating bottle apparatus) represents the amount of drug released into both luminal and abluminal regions under in vivo conditions. The amount of drug which would be released in the abluminal region is calculated by subtracting the drug released during the tube experiment from the drug released during the rotating bottle experiment at the same time.

These and other embodiments will be apparent to those of skill in the art and others in view of the following detailed description of some embodiments. It should be understood, however, that this summary, and the detailed description illustrate only some examples of various embodiments and are not intended to be limiting to the invention as claimed.

### EXAMPLES

Only a few examples and implementations are disclosed. Variations, modifications, and enhancements to the described examples and implementations and other implementations can be made based on what is disclosed.

Examples are set forth herein below and are illustrative of different amounts and types of reactants and reaction conditions that can be utilized in practicing the disclosure. It will be apparent, however, that the disclosure can be practiced with other amounts and types of reactants and reaction conditions than those used in the examples, and the resulting devices will have various properties and uses in accordance with the disclosure above and as pointed out hereinafter.

### Example 1

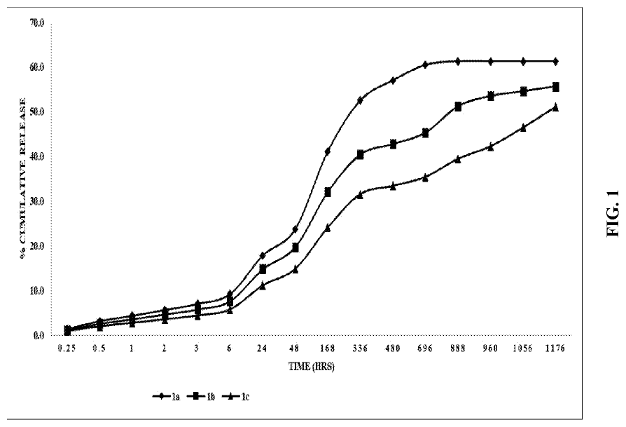

Three stents 1a, 1b, 1c were spray coated with three solutions containing the drug Tirofiban HCl and poly-ε-caprolactone polymer of intrinsic viscosities (IV) 1.07, 1.3 and 1.9, respectively. The solutions were made in Dichloromethane:Methanol (90:10 vol/vol). The polymer drug ratio was 3:1. The polymer content of the solution was (0.09% wt./vol). Tirofiban HCl loading on the stent was 75-100 g. The stents were dried and were further spray coated with poly-ε-caprolactone polymer (IV 1.07) dissolved in Dichloromethane (0.09% wt./vol). The weight of polymer coated was 250-300 μg. Stents were dried and drug release experiments were carried out using a rotating bottle apparatus (RBA) and monitored by HPLC. The cumulative drug release with respect to time is shown in FIG. 1.

### Example 2

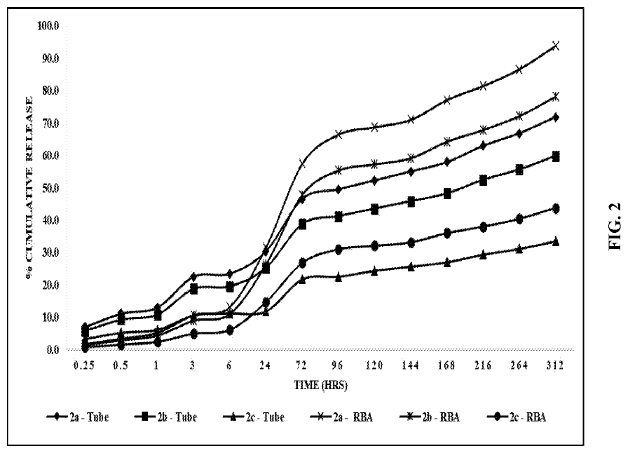

Three stents 2a, 2b, 2c were spray coated with polyvinyl pyrrolidone (K90) solutions in methanol (0.09% wt./vol polymer) containing a) Aspirin b) Tirofiban HCl and c) Clopidogrel sulphate, respectively. Polymer:drug ratio was 3:1. Drug loading was in the range 75-100 g. The stents were dried completely. Stents 2a and 2b were subsequently spray coated with poly-ε-caprolactone (PCL, IV 1.9) and stent 2c was spray coated with poly-ε-caprolactone (IV 1.3) solutions in Dichloromethane (0.09% wt./vol polymer)). The poly-ε-caprolactone loading on the stents was in the range 250-300 μg. Stents were dried, and the release of drugs was carried out using rotating bottle apparatus (RBA) and tube apparatus. Drug release was monitored by HPLC. The cumulative drug release as a function of time is shown in FIG. 2.

### Example 3

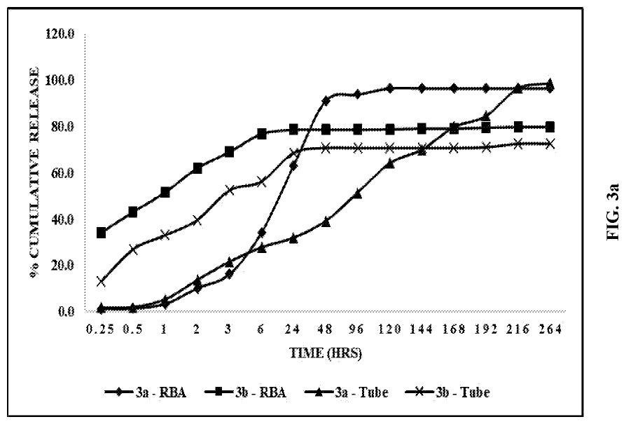

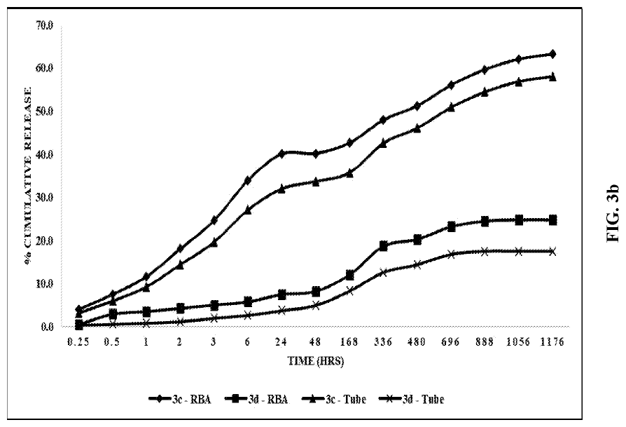

Tirofiban HCl was loaded on four stents 3a, 3b, 3c, 3d from polymer solutions wherein the polymer/Tirofiban HCl ratio was 3:1. The solution used to coat stent 3a contained polyvinyl pyrrolidone (K30) in methanol (0.09% wt./vol), stent 3b contained polyvinyl pyrrolidone (K12) in methanol (0.09% wt./vol), stent 3c contained 0.09% wt./vol. poly(D,L lactide-co glycolide) (50:50:) (IV-0.65 in HFIP, hexafluoro isopropanol) in Dichloromethane-methanol (90:10 v/v) and stent 3d contained 0.09% wt./vol, poly (L-lactide-co-s-caprolactone copolymer (80:20) (IV-1.12 in Chloroform) in Dichloromethane-Methanol (90:10 v/v). Tirofiban HCl loading on the stent was in the range 75-100 g. The stents were dried. The stents 3a and 3c were then coated using the poly-ε-caprolactone (IV 1.9 in Chloroform) solution containing (0.09% wt./vol) polymer. Stent 3b was coated with poly (D, L lactide-co glycolide) 75:25 (MW-75000) dissolved in Dichloromethane (0.09% wt./vol polymer) and stent 3d was coated with 0.09% wt./vol poly-ε-caprolactone (IV-1.07) dissolved in Dichloromethane. Stents were dried. Tirofiban HCl release experiments were carried out using rotating bottle apparatus (RBA) and tube apparatus and the release was monitored by HPLC. Cumulative release vs time is shown in FIGS. 3a and 3b

### Example 4

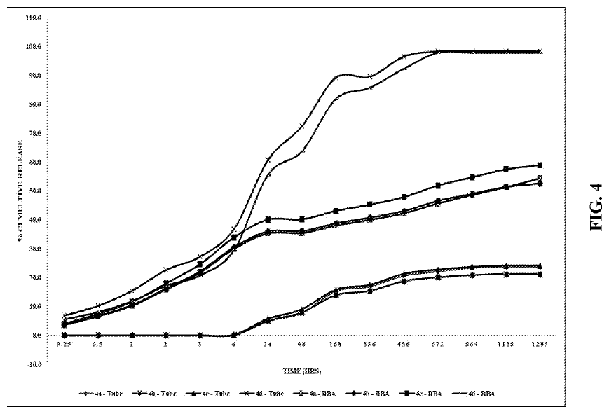

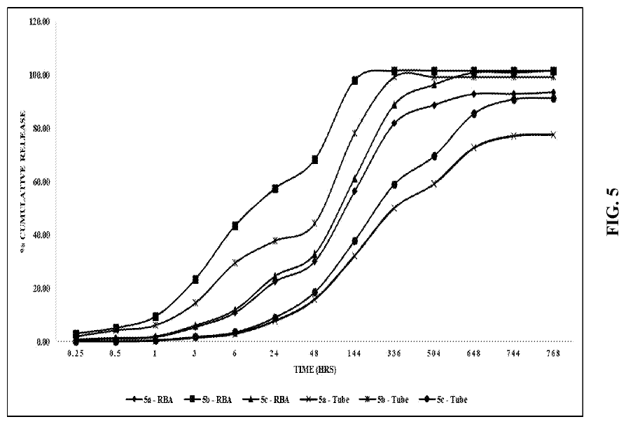

Four stents 4a, 4b, 4c, and 4d were spray coated. Stents 4a, 4b were spray coated with 0.09% wt./vol poly-ε-caprolactone (IV 1.9) dissolved in Dichloromethane. 4c was spray coated with 0.09% wt./vol poly (D, L lactide-co glycolide)copolymer 75:25 (MW-75000) dissolved in Dichloromethane and 4d was coated with 0.09% wt./vol poly-ε-caprolactone (IV 1.07) dissolved in Dichloromethane. The polymer loading was in the range 250-300 g. The stents were dried. Stent 4a was spray coated with the solution containing poly-ε-caprolactone (IV 1.07) and Everolimus (polymer-drug ratio 3:1) dissolved in dichloromethane (0.09% wt./vol polymer). Stents 4b and 4c were spray coated with the solution containing poly-ε-caprolactone (IV 1.07) and Sirolimus (polymer-drug ratio 3:1) dissolved in dichloromethane (0.09% wt./vol polymer). Stent 4d was spray coated with 0.09% wt./vol solution containing poly (D, L-lactide-co-glycolide) copolymer 75:25 (MW-75000) and Dexamethasone acetate (polymer-drug ratio 3:1 wt./wt.) dissolved in Dichloromethane-Methanol (90:10 vol/vol). Stents were dried, Drug release experiments were conducted using rotating bottle apparatus (RBA) and tube apparatus. Drug release was monitored by HPLC. Cumulative release vs time results are presented in FIG. 4.

### Example 5

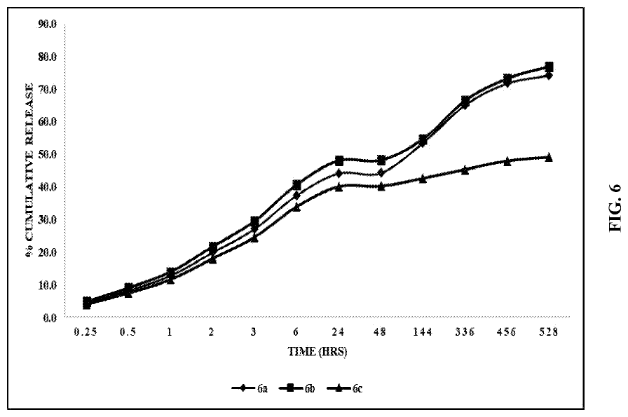

Three stents 5a, 5b and 5c were spray coated. Stent 5a was coated with drug polymer solution containing 0.09% wt./vol poly-ε-caprolactone (IV 1.07) and Everolimus (polymer-drug ratio 3:1) dissolved in Dichloromethane. Stent 5b was spray coated with drug polymer solution containing 0.09% wt./vol polymer poly-ε-caprolactone (IV 1.07) and Dexamethasone acetate (polymer-drug ratio 3:1) dissolved in Dichloromethane Methanol (90:10 vol/vol) and stent 5c was spray coated with drug polymer solution containing 0.09% wt./vol PLGA 75:25 (MW-75000) and Sirolimus (polymer-drug ratio 3:1) dissolved in Dichloromethane. Drug loading on the stents was in the range 75-100 g. The stents were dried. All three stents were further spray coated with 0.09% wt./vol poly-ε-caprolactone (IV 1.9) dissolved in Dichloromethane. Stents were dried and drug release experiments were carried out using rotating bottle apparatus (RBA) as well as tube apparatus. The release was monitored by HPLC. Cumulative drug release as a function of time is depicted in FIG. 5.

### Example 6

Three stents 6a, 6b and 6c were spray coated. Stents 6a and 6b were spray coated with 0.09% wt./vol poly-ε-caprolactone (IV 1.9) dissolved in Dichloromethane and stent 6c) was spray coated with 0.09% wt./vol PLGA 75:25 (MW-75000) dissolved in Dichloromethane. The samples were dried and were further spray coated. Stent 6a was spray coated with drug polymer solution using 0.09% wt./vol polymer dissolved in Dichloromethane, which contained poly-ε-caprolactone (IV 1.07) and poly (D, L-lactide-co-glycolide) (IV 0.65) in 1:1 weight ratio and Everolimus (polymer-drug ratio 3:1). Stent 6b was spray coated with drug polymer solution using 0.09 wt./vol polymer dissolved in Dichloromethane-methanol 90:10 vol/vol which contained poly-ε-caprolactone PCL (IV 1.07) and polyvinyl pyrrolidone (K30) in the weight ratio 1:1 and Everolimus (polymer-drug ratio 3:1). Stent 6c was coated with 0.09% wt./vol poly-ε-caprolactone (IV 1.07) dissolved in Dichloromethane and Sirolimus (polymer-drug ratio 3:1). The drug loading on the stent was in the range 75-100 μg. Stents were dried. Drug release experiments were conducted using rotating bottle apparatus (RBA) and the release was monitored by HPLC. Cumulative drug release as function of time is shown in FIG. 6.

### Example 7

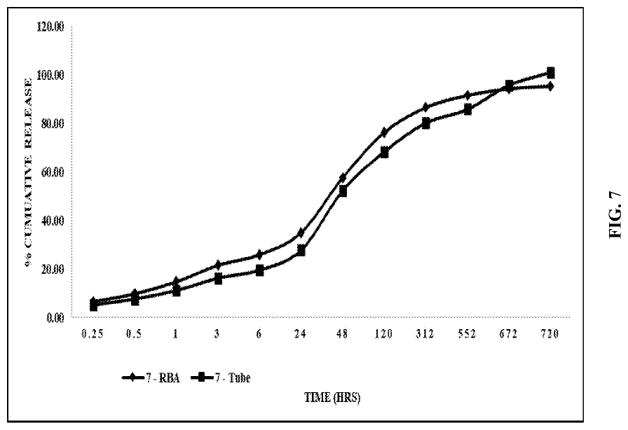

Stent 7 was spray coated with the drug polymer solution containing poly (D, L-lactide-co-glycolide) 75:25 (MW-75000) and Dexamethasone Acetate (polymer-drug ratio 3:1) dissolved in Dichloromethane (0.09% wt./vol polymer). Drug loading was 86 μg. The stent was dried and was further spray coated with 0.09% wt./vol poly-ε-caprolactone (IV 1.9) dissolved in Dichloromethane. Stent was dried and Dexamethasone Acetate release experiment was conducted using rotating bottle apparatus (RBA) and tube apparatus. The release was monitored by HPLC. Cumulative release as a function of time is shown in FIG. 7.

### Example 8

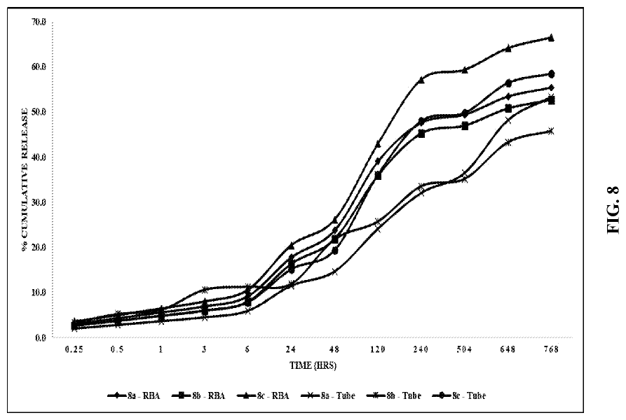

Three stents 8a, 8b and 8c were spray coated. Stent 8a was spray coated with a 0.09% wt./vol poly-ε-caprolactone (IV 1.07) in Dichloromethane-Methanol 90:10 vol/vol) and Clopidogrel (polymer-drug ratio 3:1). Stent 8b was spray coated with a 0.09% wt./vol poly-ε-caprolactone (IV 1.07) solution in Dichloromethane-Methanol (90:10 vol/vol). and Argatroban (polymer-drug ratio 3:1) Stent 8c) was spray coated with a 0.09% wt./vol poly (D, L-lactide-co-glycolide) (75:25) (MW-75000) in Dichloromethane-Methanol 90:10 vol/vol and Tirofiban·HCl (polymer-drug 3:1 ratio. Drug loading was in the range 75-100 g. Stents were dried and were further spray coated with 0.09% wt./vol poly-ε-caprolactone (IV 1.9) solution in Dichloromethane. After drying the stents, drug release experiments were carried out using rotating bottle apparatus (RBA) and tube apparatus and the drug release was monitored by HPLC. Results of cumulative drug release vs time are depicted in FIG. 8.

### Example 9

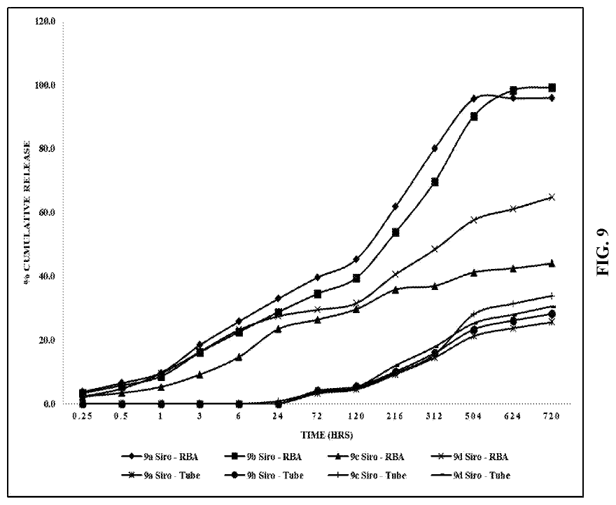

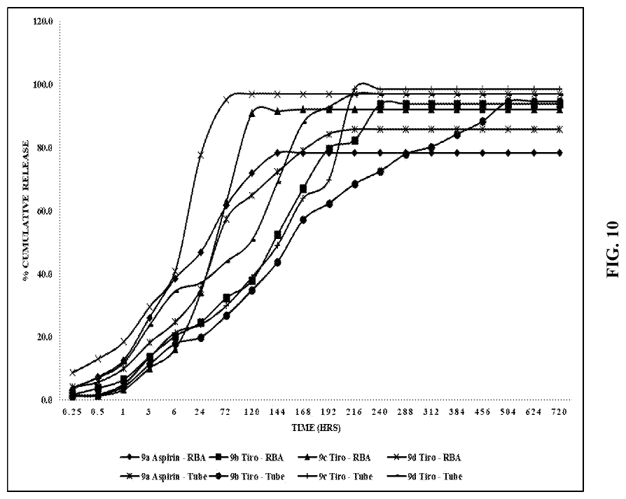

Four stents 9a, 9b, 9c and 9d were spray coated. Stent 9a was spray coated with a 0.09% wt./vol poly vinyl pyrrolidone (K90) solution in methanol and Aspirin (polymer-drug ratio 3:1). Stent 9b was spray coated with a 0.09% wt./vol poly vinyl pyrrolidone (K 90) solution in Methanol and Tirofiban HCl (polymer-drug ratio 3:1). Stent 9c was spray coated with a 0.09% wt./vol poly vinyl pyrrolidone (K 30) solution in Methanol and Tirofiban HCl (polymer-drug ratio 3:1). Stent 9d was spray coated with a 0.09% wt./vol poly vinyl pyrrolidone (K 12) solution in Methanol and Tirofiban HCl (polymer-drug ratio 3:1). Drug loading in the stents was in the range (75-100 g). Stents were dried, and all stents were spray coated with 0.09% wt./vol poly-ε-caprolactone (IV 1.9) solution in Dichloromethane. The weight of the polymer coated was in the range 250 to 300 g. Three stents except 9b were dried and further spray coated with 0.09% wt./vol poly-ε-caprolactone (IV 1.07) solution in Dichloromethane and Sirolimus (polymer-drug ratio 3:1) Stent 9b was spray coated with 0.09% wt./vol poly-ε-caprolactone (IV 1.3) solution in Dichloromethane and Sirolimus (polymer-drug ratio 3:1). The stents were dried, and the release of drugs was monitored using HPLC. Release experiment was conducted using both the rotating bottle and the tube experiment. The results of cumulative release vs time are shown in FIGS. 9 and 10.

### Example 10

Four stents 10a, 10b, 10c, and 10d were coated as follows. Stent 10a was spray coated with 0.09 wt./vol % poly (D, L-lactide-co-glycolide) (75:25, MW 75000) solution in Dichloromethane-Methanol (90:10 vol/vol) and Tirofiban hydrochloride (polymer-drug ratio, 3:1). Drug loading was 82 μg. The stent was dried. It was then spray coated with 0.09% wt./vol poly ε caprolactone (IV 1.9) solution in Dichloromethane. The polymer deposited was 262 μg. The stent was dried. It was then spray coated with a 0.09% wt./vol poly-ε-caprolactone (IV 1.3) solution in Dichloromethane and Sirolimus (polymer-drug ratio, 3:1)). Sirolimus loading was 86 μg.

Stent 10b was spray coated with a 0.09 wt./vol % poly (D, L-lactide-co-glycolide) (50:50, IV 0.65) solution in Dichloromethane-Methanol (90:10 vol/vol) and Tirofiban·HCl (polymer-drug ratio 3:1). The stent was dried. Tirofiban·HCl loading was 91 μg. It was then spray coated with a 0.09% wt./vol poly-ε-caprolactone (IV 1.9) solution in dichloromethane. The polymer deposited was 285 μg. The stent was dried again. It was spray coated with a 0.09 wt./vol % poly-ε-caprolactone (IV 1.9) solution in Dichloromethane and Sirolimus (polymer-drug ratio 3:1). Sirolimus deposited was 79 μg.

Stent 10c was spray coated with 0.09 wt./vol % poly (D, L-lactide-co-glycolide) (50:50, IV 0.65) solution in Dichloromethane-Methanol (90:10 vol/vol) and Tirofiban·HCl (polymer-drug ratio, 3:1 wt./wt.). Drug loading was 96 μg. The stent was dried. It was then spray coated with 0.09 wt./vol polymer solution in Dichloromethane containing ester terminated poly (D, L-Lactide (IV-0.65 dl/g in Chloroform) and poly-ε-caprolactone (IV 1.07) in 1:1 weight ratio. Polymer loading was 250-300 μg. The stent was dried. It was then spray coated with a 0.09 wt./vol % solution of poly-ε-caprolactone (IV 1.9) in Dichloromethane and Sirolimus (polymer-drug ratio 3:1 wt./wt.). The drug loading was 83 μg.

Stent 10d was spray coated with 0.09 wt./vol % poly (D, L-lactide-co-glycolide) (50:50, IV 0.65) solution in Dichloromethane-Methanol (90:10 vol/vol) and Tirofiban·HCl (polymer-drug ratio, 3:1 wt./wt.). Drug loading was 87 μg. The stent was dried. It was then spray coated with 0.09% wt./vol poly (L-lactide-co-s-caprolactone copolymer (80:20) (IV-1.12) in Dichloromethane. Polymer loading was 250-300 μg. The stent was dried. Further it was spray coated with 0.09 wt./vol % poly ε caprolactone (IV 1.9) solution in Dichloromethane and Sirolimus (polymer-drug ratio 3:1). Sirolimus loading was 93 μg. The stent was dried.

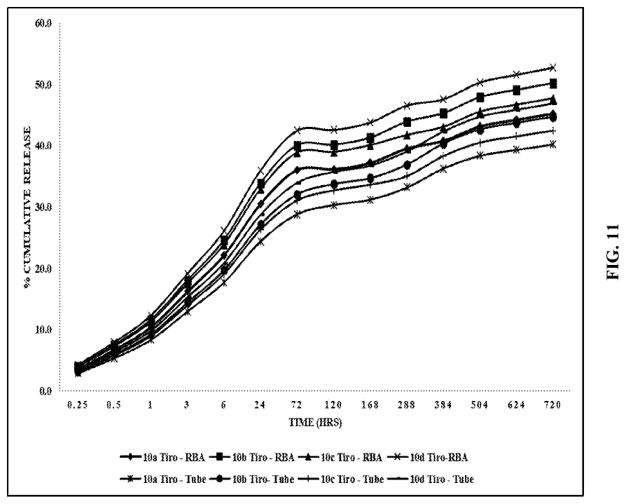

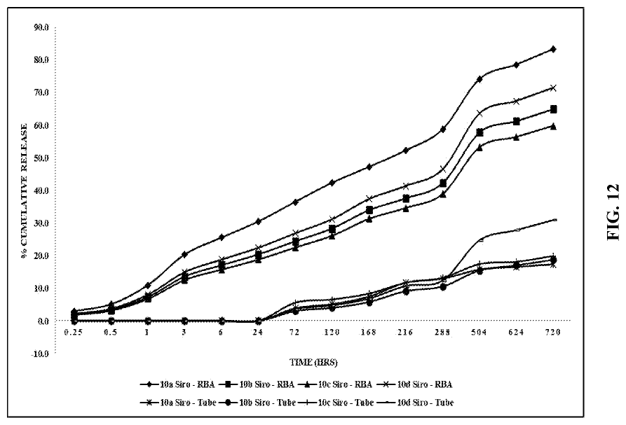

The release of Tirofiban·HCl and Sirolimus from all stents was monitored by HPLC. The release experiment was carried out using rotating bottle apparatus as well as tube experiment. Cumulative drug release vs time results are shown in FIGS. 11 and 12.

### Example 11

Three stents 11a, 11b and 11c were coated.

Stent 11a was spray coated with a solution of polyvinyl pyrrolidone (K90) and Tirofiban HCl (polymer-drug ratio 3:1) dissolved in Methanol (0.09% wt./vol polymer). Tirofiban HCl content was 42 μg. The stent was dried. It was then spray coated with a 0.09 wt./vol %) poly ε caprolactone (IV 1.07) and Aspirin (polymer-drug ratio 3:1), solution in Dichloromethane-Methanol (90:10 vol/vol). Aspirin loading was 46 g. The stent was dried. The stent was then spray coated with 0.09% wt./vol polymer) solution of poly c caprolactone (IV 1.9) in Dichloromethane. Polymer loading was 289 g. The stent was dried. Drug release experiments were conducted using rotating bottle apparatus (RBA) as well as tube apparatus and the release was monitored by HPLC.

Stent 11b was spray coated with a 0.09% wt./vol polyvinyl pyrrolidone (K90) solution in Methanol and Tirofiban HCl (polymer-drug ratio 3:1). Tirofiban·HCl content was 45 g. The stent was dried. It was then spray coated with (0.09 wt./vol %) poly ε caprolactone (IV 1.07) n in Dichloromethane-Methanol (90:10 vol/vol) and Tirofiban·HCl (polymer-drug ratio 3:1 wt./wt.). Tirofiban·HCl loading was 40 μg. The stent was dried. The stent was then spray coated with a 0.09% wt./vol poly-ε-caprolactone (IV 1.9) solution in Dichloromethane. Polymer loading was 293 μg. The stent was dried. Drug release experiments were conducted using rotating bottle apparatus (RBA) as well as tube apparatus and the release was monitored by HPLC.

Stent 11c was spray coated with a 0.09% wt./vol polyvinyl pyrrolidone (K12) solution in Methanol and Tirofiban HCl (3:1 polymer-drug ratio 3:1 wt./wt.). Tirofiban HCl loading was 48 μg. The stent was dried. It was then spray coated with a 0.09 wt./vol % poly c caprolactone (IV 1.07) solution in Dichloromethane-Methanol (90:10 vol/vol) and Tirofiban·HCl (polymer-drug ratio 3:1 wt./wt.). Tirofiban·HCl loading was 42 μg. The stent was dried. It was then spray coated with 0.09% wt./vol poly ε caprolactone (IV 1.9) solution in Dichloromethane. Polymer loading was 273 μg. The stent was dried. Drug release experiments were conducted using rotating bottle apparatus (RBA) as well as tube apparatus and the release was monitored by HPLC.

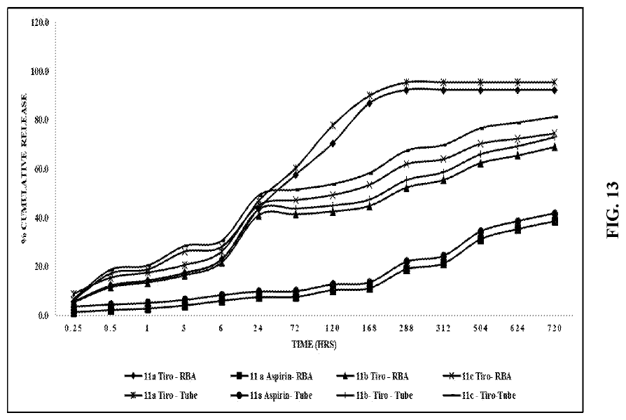

Cumulative release of drugs as a function of time for these three stents is shown in FIG. 13.

### Example 12

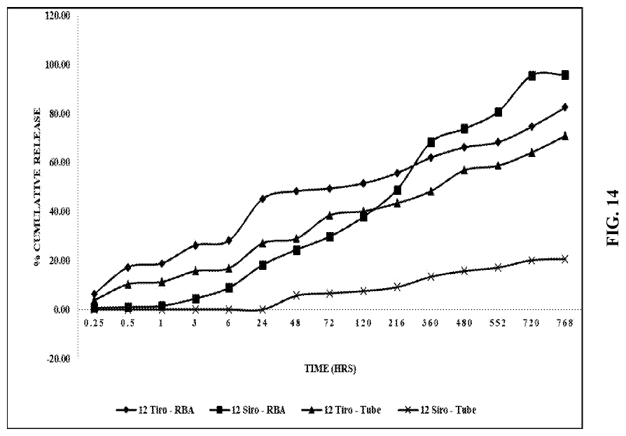

Stent 12 was spray coated with four layers of polymers. The first layer was deposited by spraying 0.09% wt./vol polyvinyl pyrrolidone (K12) solution in methanol and Tirofiban. HCl (polymer-drug ratio 3:1). Tirofiban HCl loading was (42 μg). After drying the stent, second later was deposited by spraying a 0.09 wt./vol % poly-ε-caprolactone (IV 1.07) solution in Dichloromethane-Methanol (90:10 vol/vol) and Tirofiban·HCl (polymer-drug ratio 3:1). Tirofiban HCl loading was (49 μg). A third layer was spray coated from a 0.09 wt./wt. % poly-ε-caprolactone (1.9 IV) solution in Dichloromethane. Polymer loading was 292 μg. A fourth layer was spray coated from a 0.09% wt./vol poly (D, L-lactide-co-glycolide) (75:25) (MW-75000) solution in Dichloromethane and Sirolimus (polymer-drug ratio 3:1). Sirolimus loading was (93 g)). The stent was dried. Drug release experiments were carried out using rotating bottle apparatus (RBA) and tube apparatus and the drug release was monitored by HPLC. Cumulative release of drugs as a function of time is shown in FIG. 14.

### Example 13

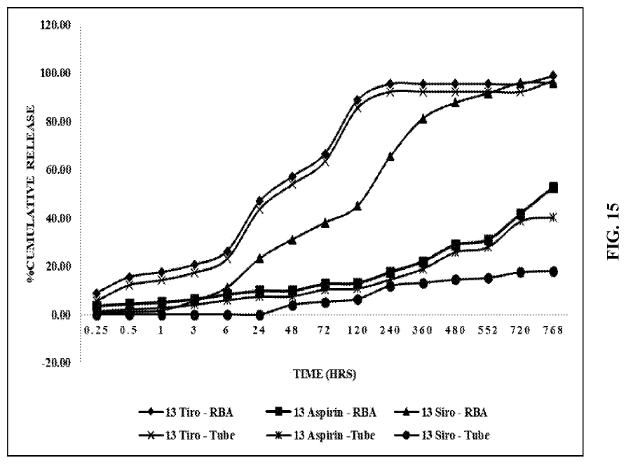

Stent 13 was spray coated with four layers of polymer. Stent was first coated with 0.09% wt./vol polyvinyl pyrrolidone (K90) solution in Methanol and Tirofiban. HCl (polymer-drug ratio 3:1). Tirofiban HCl loading was (41 μg). The stent was dried. A 0.09 wt./vol % poly ε caprolactone (IV 1.07) solution in Dichloromethane-Methanol (90:10 v/v) and Aspirin (polymer-drug ratio 3:1) was then spray coated. Aspirin loading was (39 g). The stent was dried. A third layer was spray coated from a 0.09 wt./vol % poly ε caprolactone (1.9 IV) solution in Dichloromethane. The stent was dried. A fourth layer was spray coated from a 0.09% wt./vol poly (D, L-lactide-co-glycolide) (75:25) copolymer (MW-75000) solution in Dichloromethane and Sirolimus (polymer-drug ratio 3:1). Sirolimus loading was (75-100 g). Stents were dried. Drug release experiments were carried out using rotating bottle apparatus (RBA) and the tube apparatus and the drug release was monitored by HPLC. Cumulative drug release as a function of time is shown in FIG. 15

### Example 14

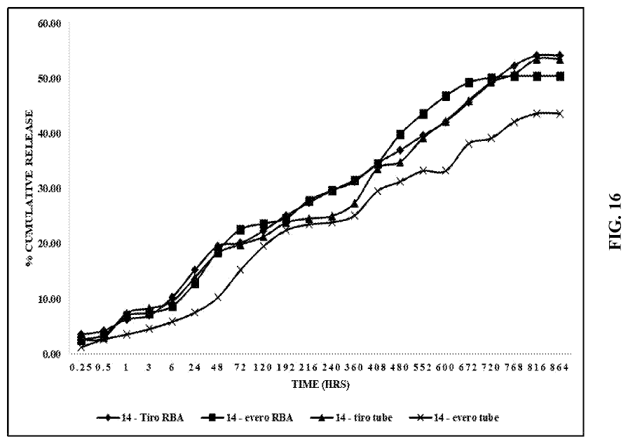

Stent 14 was spray coated with a single layer of polymer-drug composition using 0.09% wt./vol poly (L-lactide-co-caprolactone, 80:20, IV-1.12), solution in Dichloromethane and drugs used were Tirofiban HCl and Everolimus (5:3 ratio). The polymer-drug ratio was 3:1. Tirofiban HCl loading was 150 μg while Everolimus loading was 90 g. The stent was dried. Drug release experiments were carried out using rotating bottle apparatus (RBA) and the tube apparatus and the drug release was monitored by HPLC. Cumulative drug release as a function of time is shown in FIG. 16.

### Example 15

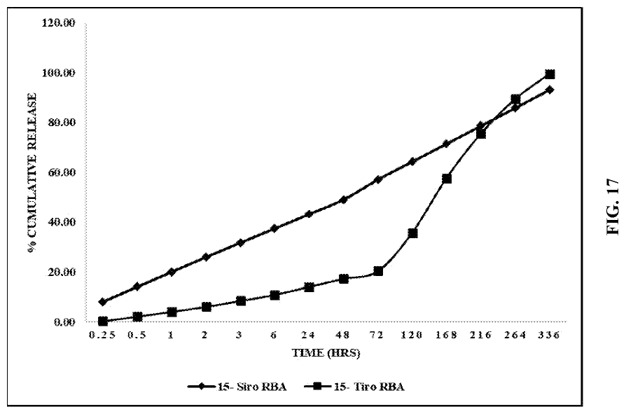

A multiple polymer layer coated stent was fabricated according to the teachings from example 10. The release data for Sirolimus and Tirofiban HCl from the rotating bottle apparatus are shown in FIG. 17. The stent was also implanted in pig model to study the effect till 28 days, sub-chronic study. Details of implantation and the histopathology observations given below.

### Animal Implantation

1. Clinically Healthy adult male Ankamali pig of 53.4 Kg body weight animal was shifted to the restraint cage 7 days prior to the day of implantation for acclimatisation and was under standard maintenance protocol.
2. First whole blood sample was collected before stent implantation for baseline haematology/Biochemistry and for evaluation of baseline coagulation parameters—platelet count, clotting time (CT) and bleeding time (BT).
3. The implantation was done under systemic heparinisation @3 mg (300 IU) per Kg BW.
4. Heparin was not reversed after the procedure.
5. No Dual Anti Platelet Therapy (DAPT) or Low molecular weight heparin was administered during the observation period.
6. Second blood sample was collected on post-operative day (follow-up on 10 days) for haematology/Biochemistry as well as evaluation of coagulation parameters such as CT, BT, APTT, ACT, and platelet count.
7. At the end of 28 days, animal was anesthetised and brought to OT.
8. Third blood sample was collected on 28th post-operative day (follow-up on 28 days) for haematology/Biochemistry as well as evaluation of coagulation parameters such as bleeding time (BT) & clotting time (CT).
9. A check angiogram was acquired under heparin (1.5 mg/Kg) to assess vessel patency.
10. Animal was euthanized using excessive dose of intravenous anaesthetic Thiopentone sodium.
11. Heart was perfusion fixed in situ.
12. A detailed necropsy was performed, and the stented vessel segments was subjected to histopathological and histomorphometry evaluation following standard protocol.

The effect of Tirofiban HCl so administered is summarized below.

| | On the day of | | |
|---|---|---|---|
| | implantation | | |
| Test stent | before | On 10^{th} | On 28^{th} |
| without | heparinisation | day post | day at |
| any DAPT | (Base line) | implantation | autopsy |
| CT sec | 185 | 245 | 240 |
| BT sec | 105 | 145 | 135 |

When Tirofiban HCl is injected intravenously, clotting time and bleeding time usually increase 3-4-fold. In the present case the increase in clotting and bleeding time was 30-40%.

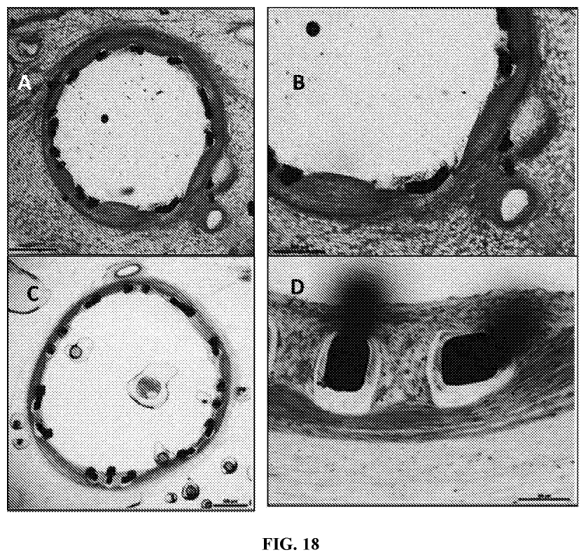

Images of implanted stent after 10 days (A, B) at the right coronary artery (RC Proximal) of swine model indicate no neointimal growth (FIG. 18). Absence of new endothelial cellular layer indicates the activity of Sirolimus in the abluminal region. Images of implanted stent after 28 days (C, D) at the right coronary artery (RC Proximal) of swine model. The growth of new endothelial cellular layer indicates absence of Sirolimus in the abluminal region. The inner surface of the stent is open to blood flow. The absence of any thrombus or clot at the end of 10 days indicates the release of tirofiban in the luminal region which prevents thrombus formation. By the end of 28 days, new cellular growth appears in the luminal region indicating the endothelialisation of the stent.

Results of histopathological and histomorphometry evaluation are shown in FIG. 18.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the scope of the present invention.

1. A multiple polymer layer coated biomedical device comprising at least one hydrophobic drug in a hydrophobic polymer layer and a hydrophobic polymer layer devoid of any drug, wherein the hydrophobic drug is released preferentially in the abluminal region.

2. A multiple polymer layer coated biomedical device as claimed in claim 1 wherein the biomedical device is selected from stents, grafts, balloons, catheters, filters or mesh like structures.

3. A multiple polymer layer coated biomedical device as claimed in claim 1, comprising a) a hydrophobic polymer layer coated on the biomedical device and b) a layer containing hydrophobic polymer and a hydrophobic drug wherein the hydrophobic drug is preferentially released in the abluminal region.

4. A multiple polymer layer coated biomedical device as claimed in claim 1, comprising a) a hydrophobic polymer layer coated on the biomedical device and b) a layer containing hydrophobic polymer and a hydrophobic drug wherein less than 20% of the hydrophobic drug is released in the abluminal region in 14 days.

5. A multiple polymer layer coated biomedical device as claimed in claim 1, comprising a) a hydrophobic polymer layer coated on the biomedical device and b) followed by a hydrophobic polymer layer coated on a) wherein less than 10% of the hydrophilic drug is released in the abluminal region in 24 hrs.

6. A multiple polymer layer coated biomedical device as claimed in claim 1, comprising a) a layer containing hydrophilic drug and a hydrophilic polymer coated on the biomedical device followed by b) a hydrophobic polymer layer coated on a) followed by c) a layer containing hydrophobic polymer and a hydrophobic drug coated on b) wherein less than 1% of hydrophobic drug is released in the luminal region up to 24 hrs.

7. A multiple polymer layer coated biomedical device as claimed in claim 6 wherein 32-74% of hydrophobic drug is released in the abluminal region in 21 days.

8. A multiple polymer layer coated biomedical device as claimed in claim 6 wherein 21-28% of hydrophobic drug is released in the luminal region in 21 days.

9. A multiple polymer layer coated biomedical device as claimed in claim 6 wherein 68-98% of hydrophilic drug is released in the luminal region in 9 days.

10. A multiple polymer layer coated biomedical device as claimed in claim 1 comprising a) a layer containing a hydrophilic drug and a hydrophobic polymer coated on the device, b) a hydrophobic polymer layer coated on a) and c) a layer containing a hydrophobic polymer and a hydrophobic drug coated on b) wherein 40-46% hydrophilic drug is released in the luminal region in 30 days.

11. A multiple polymer layer coated biomedical device as claimed in claim 10 wherein 40-65% hydrophobic drug is released in the abluminal region in 30 days.

12. A multiple polymer layer coated biomedical device as claimed in claim 1 comprising a) a layer containing a hydrophilic drug and a hydrophilic polymer coated on the biomedical device, b) a layer containing a hydrophilic drug and a hydrophobic polymer coated on a) and c) a layer containing hydrophobic polymer and a hydrophobic drug coated on b) wherein no hydrophilic drug is released in the abluminal region.

13. A multiple polymer layer coated biomedical device as claimed in claim 1 comprising a) a layer containing hydrophilic drug and a hydrophilic polymer coated on the device, b) a layer containing hydrophilic drug and a hydrophobic polymer coated on b), c) a hydrophobic polymer layer coated on b) and d) a layer containing hydrophobic drug and a hydrophobic polymer coated on c) wherein the hydrophobic drug is not released in the luminal region up to at least 24 hrs.

14. A multiple polymer layer coated biomedical device as claimed in claim 13 wherein 75% hydrophobic drug is released over 30 days in abluminal region.

15. A multiple polymer layer coated biomedical device as claimed in claim 12 wherein 64% hydrophilic drug is released in the luminal region in 30 days.

16. A multiple polymer layer coated biomedical device as claimed in claim 12 wherein the hydrophilic drug in the hydrophilic polymer layer and the hydrophilic drug in the hydrophobic polymer layer are different.

17. A multiple polymer layer coated biomedical device as claimed in claim 1 wherein the hydrophobic polymer is selected from poly-ε caprolactone (PCL), poly lactide co-ε caprolactone (PLCL), poly lactic acid (PLA), Poly lactide co-glycolide (PLGA), and blends thereof.

18. A multiple polymer layer coated biomedical device as claimed in claim 6 wherein the hydrophilic polymer is Poly vinyl pyrrolidone.

19. A multiple polymer layer coated biomedical device comprising a) a layer containing a hydrophobic polymer and a hydrophilic drug coated on the biomedical device and b) a layer containing a hydrophobic polymer blend and a hydrophobic drug coated on a) wherein the biomedical device administered to a mammal inhibits thrombus formation up to at least ten days.

20. A multiple polymer layer coated biomedical device comprising a) a layer containing a hydrophobic polymer and a hydrophilic drug coated on the biomedical device and b) a layer containing a hydrophobic polymer blend and a hydrophobic drug coated on a) wherein the biomedical device administered to a mammal inhibits neointimal growth up to at least ten days.

21. A multiple polymer layer coated biomedical device comprising a) a layer containing a hydrophobic polymer and a hydrophilic drug coated on the biomedical device and b) a layer containing a hydrophobic polymer blend and a hydrophobic drug coated on a) wherein the biomedical device administered to a mammal exhibits endothelialization between 10 and 28 days.

22. A multiple polymer layer coated biomedical device as claimed in claim 1 wherein the hydrophobic drug is selected from the class of antiproliferatives, anti-inflammatories, antibiotics, bioactive molecules, Vasodilators and Vasorelaxants.

23. A multiple polymer layer coated biomedical device as claimed in claim 22 wherein the antiproliferative drug is selected from Sirolimus (rapamycin), Everolimus, Tacrolimus, Pimecrolimus, Paclitaxel and Docetaxel and pharmaceutically acceptable salts thereof.

24. A multiple polymer layer coated biomedical device as claimed in claim 22 wherein the anti-inflammatory drug is selected from Aspirin, Celecoxib, Rofecoxib Piroxicam, Ibuprofen, Ketoprofen, Indomethacin, Diclofenac, Dexamethasone, Betamethasone, Prednisolone, Prednisone, Triamcinolone, Methyl prednisolone and pharmaceutically acceptable salts thereof.

25. A multiple polymer layer coated biomedical device as claimed in claim 1 wherein the hydrophilic drug is selected from the class of antithrombotic drugs, antiplatelet agents, thrombin inhibitors, Glycoprotein IIb/IIIa (GPIIbIIIa) inhibitors, Adenosine diphosphate (ADP) inhibitors.

26. A multiple polymer layer coated biomedical device as claimed in claim 25 wherein the anti-thrombotic drug is selected from Aspirin, Clopidogrel, Tirofiban HCl, and Argatroban and pharmaceutically acceptable salts thereof.
